(19) United States
(12) Patent Application Publication
(10) Pub. No.: US 2024/0408355 A1
(43) Pub. Date: Dec. 12, 2024

(54) ROBOTIC CATHETERISATION SYSTEM, ASSOCIATED CATHETER ROBOT, CONTROL INTERFACE AND METHOD FOR OPERATING A ROBOTIC CATHETERISATION SYSTEM

(71) Applicant: ROBOCATH, ROUEN (FR)

(72) Inventors: Cyrille Grégory COLLETTE, FRANQUEVILLE - SAINT PIERRE (FR); Fabien STRAZISAR, ROUEN (FR); Michaël ROGUE, FRANQUEVILLE - SAINT PIERRE (FR); Florian TEF, LE HOULME (FR)

(21) Appl. No.: 18/699,731

(22) PCT Filed: Oct. 10, 2022
(86) PCT No.: PCT/EP2022/078008

§ 371 Date: Apr. 9, 2024

(30) Foreign Application Priority Data
Oct. 11, 2021 (FR) 2110743

(51) Int. Cl. A61M 25/01

(52) U.S. Cl. CPC A61M 25/0116; A61M 2205/3365

(57) ABSTRACT

The invention relates to a robotic catheterization system comprising: a catheter robot (1) comprising a drive module for an elongate flexible medical instrument and a local control interface for the catheter robot (1), a remote control interface (3) for the catheter robot (1), and a communication link (8) between the catheter robot (1) and the remote control interface (3). The remote control interface (3) sends, at a first frequency, frames of movement speed settings to the catheter robot (1) via the link (8); when the catheter robot (1) no longer receives these frames for a duration exceeding a first given threshold (S1), then the drive module reduces or cancels the movement speed of the elongate flexible medical instrument for a duration exceeding a second given threshold (S3), then the drive module stops and blocks the movement of the elongate flexible medical instrument, this movement of the elongate flexible medical instrument requiring, in order to be restarted after blocking, two separate release instructions, respectively received from the remote control interface (3) and from the local control interface, the value of the second threshold (S3) being at least twice that of the first threshold (S1).

### FIELD OF THE INVENTION

The invention relates to the technical field of robotic catheterization systems, catheter robots, control interfaces, and associated methods for operating robotic catheterization systems.

### TECHNOLOGICAL BACKGROUND OF THE INVENTION

According to a first prior art, a robotic catheterization system is known which comprises a catheter robot comprising a drive module for an elongate flexible medical instrument, a control interface for the catheter robot, a link between the catheter robot and the control interface which on the one hand is over a short distance, i.e. between a few meters and several tens of meters but always less than 100 m, and which on the other hand is direct, i.e. without traveling over a communications or telecommunications network comprising switches and/or routers and/or servers or any other data or information processing devices which are intrinsically likely to cause latency in the transmission of information or data.

The control interface then allows a practitioner, for example a physician, to control the movement speed of the elongate flexible medical instrument in the catheter robot, by means of this short-distance link. This short-distance link is sufficient to protect the practitioner from radiation, in particular from X-ray radiation, while enabling him or her to continue to diagnose and/or treat the patient in a safe and effective manner, provided that he or she remains close to the patient, typically in a neighboring room in the same hospital (or at least behind a protective lead screen, if the practitioner remains in the same room), and that the link between control interface and catheter robot remains direct.

According to a second prior art, a robotic catheterization system is known which comprises a catheter robot comprising a drive module for the translational movement of an elongate flexible medical instrument, a remote control interface for the catheter robot, and a long-distance link, meaning over a distance greater than 10 km or greater than 50 km, between the catheter robot and the remote control interface.

The remote control interface then allows a practitioner, for example a physician, to control the movement speed of the elongate flexible medical instrument in the catheter robot, by means of this long-distance link. This long-distance link is not only more than sufficient to protect the practitioner from radiation, in particular from X-ray radiation, while enabling him or her to continue to diagnose and/or treat the patient, but it allows the practitioner to continue to diagnose and/or treat the patient over a long distance, even from another hospital, and even from a hospital located in another region. In this case, a less experienced practitioner, who is at least a practitioner with paramedical or nursing skills, is on site next to the catheter robot as a precautionary measure, in order to be able to manipulate the local control interface of the catheter robot if necessary and possibly also as ordered by the more experienced practitioner (over a cell phone link for example).

This allows an experienced or very specialized practitioner to be able to:
intervene effectively even if a patient requiring urgent treatment is far from the hospital where this very specialized practitioner is located, which can be of inestimable help particularly when the medical condition is a difficult one and the intervention of an experienced practitioner is, if not necessary, at least highly useful,
and to more easily provide for such difficult interventions in the event of non-urgent situations, without necessarily requiring the patient to be moved and hospitalized in the hospital where the experienced practitioner works.
Unfortunately, the long distance between the experienced practitioner and the patient, as well as the possibility of various switches and/or routers and/or servers or any other data or information processing devices which are inherently likely to cause latency in the transmission of information or data, in a telecommunications network to be traveled by the commands sent by the remote control interface from the practitioner to the catheter robot located near the patient, makes this type of surgery slow and tricky, therefore ineffective or even dangerous if the surgery is complex, due to the significant latency resulting from the time required for the commands to travel between practitioner and patient, thus losing much of the advantages of remote surgeries by reducing them to simple surgeries that do not necessarily have to take place in critical real time.

### OBJECTS OF THE INVENTION

The aim of the present invention is to provide a robotic catheterization system that at least partially overcomes the above disadvantages.

One way to improve this situation would be to greatly accelerate the travel time on very fast and very secure direct transmission lines, in particular not traveling over any conventional communication or telecommunications network that would include various switches and/or routers and/or servers or any other data or information processing devices inherently likely to cause latency in the transmission of information or data.

Rather, the invention proposes another approach which is simpler and less expensive, whether for a short-distance link or for a long-distance link, consisting of using a more conventional communication or telecommunications network but still protecting the operation of the catheter robot in the event of too much latency, or even in the event of a loss of transmission of the commands sent via the remote control interface by the practitioner to the catheter robot located near the patient.

To do this, the catheter robot will react to protect the surgical procedure and maintain the integrity of the patient in a graduated manner according to the increase in latency time, while continuing to be able to use its link to its remote control interface, or even to use an ordinary or conventional network, possibly the Internet, while ensuring a good compromise between the safety of the remote surgery and the fluidity of the remote surgery, taking into account any particular circumstances of this remote surgery such as lag or a temporary loss of data transmission.

First of all, the catheter robot will reduce or even cancel the movement speed of the elongate flexible medical instrument in the catheter robot, and then it will block this movement of the elongate flexible medical instrument in the catheter robot, advantageously by entering a robot error mode which will require a first release instruction preferably entered directly at the local control interface in order to be able to continue this movement of the elongate flexible medical instrument in the catheter robot.

In addition, and again as a safety measure, the remote control interface, after having also advantageously placed itself in remote interface error mode, will preferably also have to send back a second release instruction via the link, obtained by a specific action by the user of the remote control interface, such as releasing the joystick (if the remote control interface is a joystick) or such as pressing a restart or reset button, in order to be able to continue this movement of the elongate flexible medical instrument in the catheter robot.

Safety is not only improved, it is greatly improved, even almost maximized, to the extent that the catheter robot will perform a double verification before being able to continue this movement of the elongate flexible medical instrument in the catheter robot:
first verification: the catheter robot is once again locally ready to restart this movement of the elongate flexible medical instrument in the catheter robot,
second verification: the remote control interface is once again ready to remotely control the catheter robot in order to restart this movement of the elongate flexible medical instrument in the catheter robot.

According to the invention, a robotic catheterization system is provided which comprises: a catheter robot comprising a drive module for an elongate flexible medical instrument and a local control interface for the catheter robot, a remote control interface for the catheter robot, and a communication link between the catheter robot and the remote control interface, the remote control interface enabling a practitioner to control the movement speed of the elongate flexible medical instrument in the catheter robot by means of the link, characterized in that: the remote control interface sends, at a first frequency, frames of movement speed settings to the catheter robot via the link; when the catheter robot receives these frames, then the drive module moves the elongate flexible medical instrument at the movement speed setting received in these frames; when the catheter robot no longer receives these frames: for a duration exceeding a first given threshold, then the drive module reduces or cancels the movement speed of the elongate flexible medical instrument relative to the last speed setting received; for a duration exceeding a second given threshold that is greater than the first threshold, then the drive module stops and blocks the movement of the elongate flexible medical instrument, this movement of the elongate flexible medical instrument requiring, in order to be restarted after blocking, a first release instruction received from the remote control interface, and a second release instruction received from the local control interface; the value of the second threshold being at least twice that of the first threshold, or at least three times that of the first threshold.

According to the invention, also provided is a catheter robot of a robotic catheterization system, comprising a drive module for an elongate flexible medical instrument and a local control interface for the catheter robot, and configured to be controlled by a remote control interface for the catheter robot enabling a practitioner to control the movement speed of the elongate flexible medical instrument in the catheter robot by means of a communication link between the catheter robot and the remote control interface, characterized in that: when the catheter robot receives frames of movement speed settings from the remote control interface via the link, then the drive module moves the elongate flexible medical instrument at the movement speed setting received in these frames; when the catheter robot no longer receives these frames: for a duration exceeding a first given threshold, then the drive module reduces or cancels the movement speed of the elongate flexible medical instrument relative to the last speed setting received; for a duration exceeding a second given threshold that is greater than the first threshold, then the drive module stops and blocks the movement of the elongate flexible medical instrument, this movement of the elongate flexible medical instrument requiring, in order to be restarted after blocking: a first release instruction received from the remote control interface, and a second release instruction received from the local control interface, the value of the second threshold being at least twice that of the first threshold, or at least three times that of the first threshold.

According to the invention, also provided is a control interface for a robotic catheterization system comprising a catheter robot comprising a drive module for an elongate flexible medical instrument, configured to enable a practitioner to control the movement speed of the elongate flexible medical instrument in the catheter robot by means of a link between the catheter robot and the control interface, the control interface being intended to be remote from the catheter robot, characterized in that the remote control interface: sends frames of movement speed settings to the catheter robot, at a first frequency, via the link; receives, from the catheter robot at a second frequency, via the link, status frames representative of the frames of speed settings received at a second frequency; so that, when the remote control interface receives these status frames, then the remote control interface continues to send frames of speed settings, and so that, when the remote control interface no longer receives these status frames: for a duration exceeding a third given threshold, then the remote control interface continues to send frames of speed settings; for a duration exceeding a fourth given threshold that is greater than the third threshold, then the remote control interface triggers a remote interface alarm perceptible to the user of the remote control interface, the value of the fourth threshold being at least twice that of the third threshold, or at least three times that of the third threshold.

According to the invention, a method is also provided for operating a robotic catheterization system comprising a catheter robot which comprises a drive module for an elongate flexible medical instrument and a local control interface for the catheter robot, and comprising a remote control interface for the catheter robot that is connected to the catheter robot via a link, such that the remote control interface enables a practitioner to control the movement speed of the elongate flexible medical instrument in the catheter robot by means of the link, characterized in that: the remote control interface sends frames of movement speed settings to the catheter robot at a first frequency, via the link; when the catheter robot receives these frames, then the drive module moves the elongate flexible medical instrument at the movement speed setting received in these frames; when the catheter robot no longer receives these frames: for a duration exceeding a first given threshold, then the drive module reduces or cancels the movement speed of the elongate flexible medical instrument relative to the last speed setting received; for a duration exceeding a second given threshold that is greater than the first threshold, then the drive module stops and blocks the movement of the elongate flexible medical instrument, this movement of the elongate flexible medical instrument requiring, in order to be restarted after blocking, a first release instruction received from the remote control interface and a second release instruction received from the local control interface, the value of the second threshold being at least twice that of the first threshold, or at least three times that of the first threshold.

According to preferred embodiments of the invention, a robotic catheterization system is proposed in which the remote control interface and the robot may each be in different locations and be separated by several hundred kilometers. The patient and the physician who operates on the patient may thus be in different hospitals, the patient being for example in a small local hospital which is the hospital closest to the place where patient's injury occurred, and the physician is in a central hospital which is large and which is located several hundred kilometers from the place where the patient's injury occurred. In this manner, remote training with the robot may also be offered for complex cases, or it may also be possible to carry out remote surgeries even in non-emergency cases.

According to preferred embodiments of the invention, a solution is proposed which may be applied to existing robotic catheterization systems and which thus does not require modifying the software of the remote control interface and of the robot (or only very slightly). In particular, it will be advantageous to be able to retain the existing control system for the link between the remote control interface and the robot, whereas this system is adapted for a link via a data bus with an immediate transmission of data without latency (typically a latency of less than 1 ms). The control system for the link between the remote control interface and the robot will be adapted to allow latency in the transmission of data, advantageously without modifying the software for the robot and of the remote control interface which controls the link between the remote control interface and the robot.

According to preferred embodiments, the invention comprises one or more of the following features which may be used separately or in a combination of some of them or in a combination of all of them, with any of the above objects of the invention.

Preferably, the link is a long-distance link, meaning at least 100 m or at least 1 km or at least 10 km or at least 50 km, between the catheter robot and the remote control interface.

The proposed invention is all the more attractive when the risk of excessive latency becomes significant, which is the case in particular for a long-distance link.

Preferably, a short-distance link, meaning at least Im but at most 100 m, between the catheter robot and the remote control interface.

Preferably, said link is over a communications or telecommunications network comprising switches and/or routers and/or servers and/or repeaters.

The proposed invention is all the more attractive when the risk of excessive latency becomes significant, which is the case in particular when traveling over multiple information processing and/or transmission devices found throughout a communications or telecommunications network which can become relatively complex.

In a first preferred embodiment, the movement speed setting is a setting for a translational movement speed.

In a second preferred embodiment, the movement speed setting is a setting for a rotational movement speed.

In a third preferred embodiment, the movement speed setting comprises both a setting for a translational movement speed and a setting for a rotational movement speed.

Thus, prolonging a translational movement of the elongate flexible medical instrument in the patient's body to travel to an unanticipated location can very quickly pose a major risk to the safety of the patient, while simply prolonging a rotation which is no longer desired will only pose a notable threat in the longer term (if the rotation is maintained for a long time, it can irritate the walls of the patient's arteries or veins, or can even enter the wrong branch in the patient's blood circulation system, when combined with a translational movement of the elongate flexible medical instrument, which initially will require backing up but which initially will not pose a risk of perforating the wall of an artery or vein of the patient.

The movement speed setting may also comprise a translational movement speed setting and a rotational movement speed setting. In this case:
losing both movement speed settings, translational and rotational, results in reducing and/or cancelling these movement speed settings, translational and rotational,
losing one of the movement speed settings, translational and rotational, results in reducing and/or cancelling:
   preferably both movement speeds, translational and rotational, as a precaution and a safety measure,
   or possibly only the movement speed, translational or rotational, corresponding to the lost speed setting, translational or rotational, if there is no risk associated with continuing one of the movements, translational or rotational, without the other movement: this may depend on the scenario envisaged, and this could even mean having different operating modes that are configurable by the user of the robotic catheterization system.

Preferably, when the catheter robot no longer receives these frames for a duration exceeding a first given threshold, then the remote control interface also triggers a remote control interface alarm perceptible to the user of this remote control interface.

The malfunction of the transmission between the remote control interface and the catheter robot is thus signaled as early as possible, even before or at the same time as appropriate reactions are triggered at the control interface and at the catheter robot.

In a preferred embodiment, when the catheter robot no longer receives these frames for a duration exceeding the first given threshold, then the drive module reduces and cancels the movement speed of the elongate flexible medical instrument, for a duration that is less than 10% of the first threshold.

Thus, the movement of the elongate flexible medical instrument in the catheter robot is stopped very quickly, which further improves patient safety.

In another preferred embodiment, when the catheter robot no longer receives these frames for a duration exceeding the first given threshold, then the drive module reduces the movement speed of the elongate flexible medical instrument relative to the last speed settings received, progressively for a duration that is greater than the first threshold or greater than twice the first threshold.

Thus, the movement of the elongate flexible medical instrument in the catheter robot is stopped relatively quickly, but without undue haste, which improves the fluidity of the operation and the resumption of movement of the elongate flexible medical instrument in the catheter robot in the event that new frames of speed settings are received after a short period of not receiving these frames, while still ensuring patient safety in a very satisfactory manner.

Preferably, when the catheter robot no longer receives these frames for a duration exceeding the first given threshold, then the drive module reduces and cancels the movement speed of the elongate flexible medical instrument relative to the last speed setting received, progressively for a duration that is greater than the first threshold or greater than twice the first threshold. In a less preferred optional embodiment, when the catheter robot no longer receives these frames for a duration exceeding the first given threshold, then the drive module could reduce the movement speed of the elongate flexible medical instrument without completely canceling it, for example reducing it by at least 80% or at least 90% or at least 95% or at least 99%, relative to the last speed setting received, for a duration greater than the first threshold or greater than twice the first threshold.

Thus, complete cancellation of the movement speed of the elongate flexible medical instrument in the catheter robot, after reducing this speed, further improves patient safety.

In a first option of this other preferred embodiment, when the catheter robot no longer receives these frames for a duration exceeding the first given threshold, then the drive module progressively and linearly reduces the movement speed of the elongate flexible medical instrument relative to the last speed setting received, for a duration greater than the first threshold or greater than twice the first threshold.

Thus, good fluidity in the operation and in the resumption of movement of the elongate flexible medical instrument in the catheter robot is obtained in the event that new frames of speed settings are received after a short period of not receiving these frames, while still ensuring patient safety in a satisfactory manner.

In a second option of this other preferred embodiment, when the catheter robot no longer receives these frames for a duration exceeding the first given threshold, then the drive module reduces the movement speed of the elongate flexible medical instrument relative to the last speed setting received, progressively and increasingly more, advantageously in a convex parabola, for a duration that is greater than the first threshold or greater than twice the first threshold.

Thus, very good fluidity is obtained in the operation and in the resumption of movement of the elongate flexible medical instrument in the catheter robot in the event of receiving new frames of speed settings after a short period of not receiving these frames, while still ensuring patient safety in a rather satisfactory manner.

In a third option of this other preferred embodiment, when the catheter robot no longer receives these frames for a duration exceeding the first given threshold, then the drive module reduces the movement speed of the elongate flexible medical instrument relative to the last speed setting received, progressively and increasingly less, advantageously in a concave parabola, for a duration greater than the first threshold or greater than twice the first threshold.

Thus, a fairly good fluidity is obtained in the operation and in the resumption of movement of the elongate flexible medical instrument in the catheter robot in the event of receiving new frames of speed settings after a short period of not receiving these frames, while still ensuring patient safety in a very satisfactory manner.

Preferably, the catheter robot sends status frames for the catheter robot that are representative of the last frames of speed settings received, to the remote control interface at a second frequency, via the link; when the remote control interface receives these status frames, then the remote control interface continues to send frames of speed settings, but when the remote control interface no longer receives these status frames: for a duration exceeding a third given threshold, then the remote control interface continues to send frames of speed settings; for a duration exceeding a given fourth threshold that is greater than the third threshold, then the remote control interface triggers a remote interface alarm perceptible to the user of the remote control interface, the value of the fourth threshold being at least twice that of the third threshold, or at least three times that of the third threshold.

Thus, in the event of a loss of communication between the remote control interface and the catheter robot, not only is the operation of the catheter robot slowed down and/or stopped, thus ensuring patient safety, but because the remote control interface is now warned of this loss of communication, the remote control interface can react in an appropriate manner, while enabling the practitioner manipulating this remote control interface to be aware of this loss of communication. The practitioner is a user of the robotic catheterization system, the one who is carrying out the remote surgery from the remote control interface.

At the catheter robot, the speed settings, which are no longer sent by the remote control interface, are replaced, for example by the robot control unit associated with the catheter robot. Thus, an untimely triggering of an alarm at the catheter robot is avoided, since this catheter robot is “deceived” and believes that the communication is still working correctly between the control interface and the catheter robot. To remain effective, this “deception” must only last a limited time, because it then becomes useful to trigger an alarm if the loss of communication between the control interface and the catheter robot persists.

Preferably, the third threshold is equal to the first threshold, and/or the fourth threshold is equal to the second threshold.

Synchronization between the catheter robot and the remote control interface is thus further improved, but slight differences between the thresholds remain conceivable and possible without impacting the effectiveness of the system, although still complicating the management of all the different thresholds.

Preferably, the status frames successively returned are numbered relative to each other, so that the remote control interface can reconstruct their chronology.

The effect of a loss of communication between the control interface and the catheter robot is thus further reduced.

Preferably, as the link of the robotic catheterization system is a long-distance link, meaning at least 100 m or at least 1 km or at least 10 km or at least 50 km, between the catheter robot and the remote control interface, the system also comprises: an instrumentation and control unit associated with the remote control interface for the catheter robot, comprising a converter which converts data in a local format interpretable by the remote control interface into packets in a long-distance format which can be sent over the long-distance link; a robot control unit associated with the catheter robot, comprising a converter which converts packets in a long-distance format that are received from the long-distance link into data in a local format interpretable by the catheter robot; the instrumentation and control unit and the robot control unit respectively being located at the two ends of the long-distance link.

Thus, the presence of control units and associated converters makes it possible to maintain a short-distance communication protocol dedicated to the operation of medical devices such as the control interface and the catheter robot, while using a long-distance communication protocol more suitable for sending information over a long distance between the remote control interface and the catheter robot.

Preferably, the instrumentation and control unit also comprises a converter which converts packets in a long-distance format that are received from the long-distance link into data in a local format interpretable by the remote control interface; the robot control unit also comprises a converter which converts data in a local format interpretable by the catheter robot into packets in a long-distance format which can be sent over the long-distance link.

The long-distance communication protocol can thus work in both directions.

Preferably, the second threshold is chosen such that the elongate flexible medical instrument cannot travel more than 10 mm in translational movement, after having stopped receiving the frames of speed settings, even at its maximum speed of translational movement.

Patient safety is thus ensured, while maintaining sufficient fluidity in the operation of the catheter robot.

Preferably, the second threshold is chosen such that the elongate flexible medical instrument cannot rotate more than 360° after having stopped receiving the frames of speed settings, even at its maximum speed of rotational movement.

Patient safety is thus ensured, while maintaining sufficient fluidity in the operation of the catheter robot.

Preferably, the first threshold is between 10 ms and 200 ms, or between 50 ms and 150 ms, or is 100 ms.

These first threshold ranges ensure good optimization of patient safety, while maintaining good fluidity in the operation of the catheter robot.

Preferably, the second threshold is between 200 ms and 5000 ms, or between 400 ms and 2000 ms, or is 1000 ms.

These second threshold ranges ensure good optimization of patient safety, while maintaining good fluidity in the operation of the catheter robot.

Preferably, the duration during which the movement speed of the elongate flexible medical instrument is reduced relative to the last speed setting received, until it is canceled, is between 10 ms and 300 ms, or between 100 ms and 200 ms, or is 150 ms.

These duration ranges for reducing the movement speed of the elongate flexible medical instrument ensure good optimization of patient safety, while maintaining good fluidity in the operation of the catheter robot.

Preferably, the robotic catheterization system also comprises a medical imaging device which is associated with the catheter robot, located on the same side of the link as the catheter robot, connected to the catheter robot by a local communication bus.

The practitioner can thus monitor the movement of the elongate flexible medical instrument inside the patient's body in real time, which allows the practitioner to react immediately, if necessary, at the remote control interface.

Preferably, the local communication bus is a CAN bus.

CAN is a standard and is an acronym for “Control Area Network”.

This communication bus is particularly well suited for supporting a short-distance communication protocol between medical devices, such as the local control interface, the catheter robot, and possibly the medical imaging device.

Preferably, the long-distance link transmits data packets in TCP/IP format or in UDP/IP format.

TCP/IP is a standard and is an acronym for Transfer Control Protocol/Internet Protocol.

UDP/IP is a standard and is an acronym for User Data Protocol/Internet Protocol.

These long-distance communication protocols are particularly well suited for rapid, virtually lossless transmission over long distances, such as between the remote control interface and the catheter robot.

Preferably, the long-distance link traverses a WAN communications network.

A WAN network is a network of at least regional extent and stands for “Wide Area Network”.

Traversing a telecommunications network, in addition to traveling over a long-distance link, makes the invention all the more attractive as the risk of lag or a loss of communication between the remote control interface and catheter robot increases with:
the length of the distance to be covered,
and the complexity of the telecommunications network to be traversed.

Preferably, the first frequency is between 1 Hz and 1 kHz, or between 10 Hz and 500 Hz, or between 50 Hz and 200 Hz, or is 100 Hz, and/or the first frequency is constant, and/or the second frequency is between 1 Hz and 1 kHz, or between 10 Hz and 500 Hz, or between 50 Hz and 200 Hz, or is 100 Hz, and/or the second frequency is constant.

These relatively high frequency ranges thus provide dual advantages:
the frequencies, variable (quasi-periodic signal for example) or constant (periodic signal), are high enough to allow almost immediately securing the safety of the patient,
the frequencies, variable or constant, are sufficiently moderate to avoid disrupting or blocking the operation of the catheter robot unintentionally or a bit too often, and to prevent the catheter robot from being stopped too often.

Preferably, the alarm is visual, or the alarm is audio, or the alarm is both visual and audio.

The practitioner at the remote control interface will thus be more likely to realize more quickly that there is a malfunction in the robotic catheterization system.

Preferably, when the catheter robot once again receives these frames after no longer receiving these frames for some time, the drive module then moves the elongate flexible medical instrument at the movement speed setting received in the frame sent most recently by the remote control interface.

Thus, fluidity in the operation of the catheter robot is further improved, without sacrificing patient safety.

Preferably, the frames of movement speed settings sent successively are numbered relative to each other, so that the catheter robot can reconstruct their chronology.

Once communication resumes working correctly between the remote control interface and the catheter robot, the catheter robot will thus be able to take into account the speed setting sent most recently by the remote control interface. The effect of the loss of communication between the remote control interface and the catheter robot is thus further reduced.

Preferably, the elongate flexible medical instrument includes a catheter and/or a catheter guide.

Preferably, after the first threshold has been exceeded, the remote control interface must return to the zero speed setting by an action by the user of the remote control interface, to enable the remote control interface to regain control of the catheter robot.

After exceeding the first threshold, the operator must reset the remote control interface to zero, in order to be able to regain control of the catheter robot and thus be able to return to normal mode, in order to avoid overcompensating for the lag.

In other words, there are two conditions in order to return from degraded mode (first threshold exceeded) to normal mode:
the first condition is that the latency must drop back below the first threshold,
and the second condition is that the user of the robotic catheterization system, for example the physician or practitioner, releases the remote control interface, for example the joystick, for a predetermined duration, or restarts or resets it in another manner, such as by pressing a restart or reset button.

Preferably, the catheter robot is configured to send a latency measurement message at a third frequency to the remote interface via the link, said remote interface being configured to send said latency measurement message back to the catheter robot upon receipt, said catheter robot being configured to measure the duration between transmission and reception of the latency measurement message by said catheter robot, said catheter robot being configured so that the drive module stops and blocks the movement of the elongate flexible medical instrument when the duration between transmission and reception of the latency measurement message by said catheter robot exceeds a predetermined value.

Such latency measurement helps improve safety by monitoring another aspect of link quality.

According to an additional object of the invention, a robotic catheterization system is provided comprising: a catheter robot comprising a drive module for an elongate flexible medical instrument and a local control interface for the catheter robot; a remote control interface for the catheter robot; and a communication link between the catheter robot and the remote control interface; the remote control interface enabling a practitioner to control the movement speed of the elongate flexible medical instrument in the catheter robot by means of the link; also comprising one or more of the following remote maintenance functionalities:
searching for a malfunction or bug:
   in the catheter robot, from the remote control interface,
   and/or in the remote control interface, from the local interface of the catheter robot,
and/or performing diagnostics on operations:
   for the catheter robot, from the remote control interface,
   and/or for the remote control interface, from the local interface of the catheter robot,
and/or downloading an update:
   to the catheter robot software, from the remote control interface,
   and/or to the software of the remote control interface, from the local interface of the catheter robot,
and/or updating operating parameters:
   of the catheter robot, from the remote control interface,
   and/or of the remote control interface, from the local interface of the catheter robot.

This additional object of the invention may also be combined:
with one or more of the aforementioned preferred features which may be used separately or in a combination of some of them or in a combination of all of them,
and/or with any of the aforementioned objects of the invention.

Other features and advantages of the invention will become apparent upon reading the following description of a preferred embodiment of the invention, given by way of example and with reference to the appended drawings.

### BRIEF DESCRIPTION OF DRAWINGS

FIG. 1 schematically represents an example of a robotic catheterization system according to one embodiment of the invention.

FIG. 2 schematically represents a first example of a speed profile which corresponds to a first type of reaction of the catheter robot following the loss of reception of the speed setting sent by the remote control interface, in an example of a robotic catheterization system according to one embodiment of the invention.

FIG. 3 schematically represents a second example of a speed profile which corresponds to a second type of reaction of the catheter robot following the loss of reception of the speed setting sent by the remote control interface, in an example of a robotic catheterization system according to one embodiment of the invention.

FIG. 4 schematically represents a third example of a speed profile which corresponds to a third type of reaction of the catheter robot following the loss of reception of the speed setting sent by the remote control interface, in an example of a robotic catheterization system according to one embodiment of the invention.

FIG. 5 schematically represents a fourth example of a speed profile which corresponds to a fourth type of reaction of the catheter robot following the loss of reception of the speed setting sent by the remote control interface, in an example of a robotic catheterization system according to one embodiment of the invention.

### DETAILED DESCRIPTION OF EMBODIMENTS OF THE INVENTION

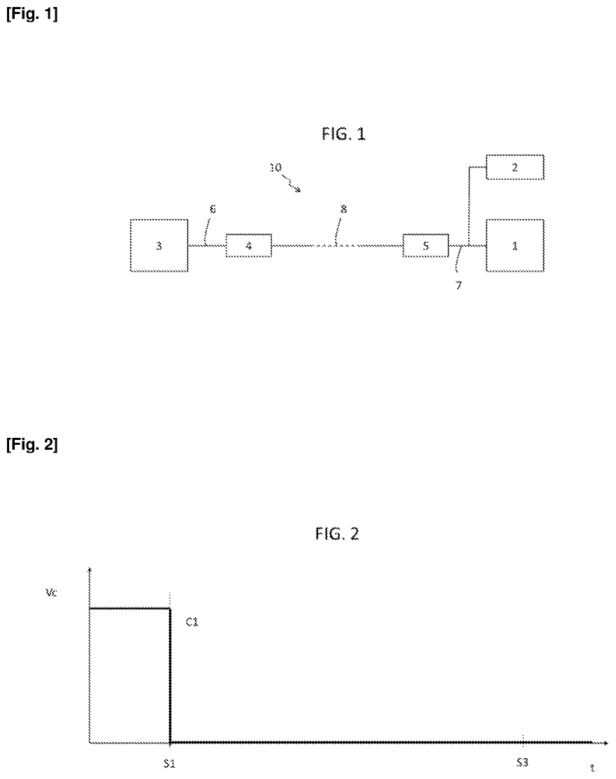

FIG. 1 schematically represents an example of a robotic catheterization system according to one embodiment of the invention.

A robotic catheterization system 10 comprises: a catheter robot 1 which itself comprises a drive module for driving the translational movement of an elongate flexible medical instrument and a local interface for controlling the catheter robot, a remote control interface 3 for catheter robot 1, a communication link 8 between catheter robot 1 and remote control interface 3, remote control interface 3 enabling a practitioner to control the movement speed of the elongate flexible medical instrument in catheter robot 1 by means of link 8. Robot 1 may also comprise a drive module for driving the rotational movement, or a module capable of driving both the translational and rotational movement of the medical instrument.

Catheterization robot 1 is intended to control the movements of various flexible and elongate medical instruments inside the body of a patient. The elongate flexible medical instruments may for example be a guide catheter, a catheter, and a catheter guide, or a combination of these elements.

Remote control interface 3 enables the physician to control the movements of robot 1 in order to control the movements of the different flexible and elongate medical instruments.

Remote control interface 3 sends, at a first frequency, frames of movement speed settings to catheter robot 1, via link 8.

When catheter robot 1 receives these frames, then the drive module moves the elongate flexible medical instrument at the movement speed setting received in these frames.

When catheter robot 1 no longer receives these frames for a duration exceeding a first given threshold S1, then the drive module reduces or cancels the movement speed of the elongate flexible medical instrument relative to the last speed setting received.

When catheter robot 1 no longer receives these frames for a duration exceeding a second given threshold S3 that is greater than the first threshold S1, then the drive module stops and blocks the movement of the elongate flexible medical instrument.

Once blocked, this movement may be restarted without repeating the entire initialization process that precedes a new remote surgery. However, in order to be restarted after blocking, this movement of the elongate flexible medical instrument will then require two separate release instructions which are:
a first release instruction received from remote control interface 3,
and a second release instruction received from the local control interface of catheter robot 1.

The value of the second threshold S3 is advantageously at least three times that of the first threshold S1.

When catheter robot 1 once again receives these frames after no longer receiving the frames for a time that is less than the first threshold S1, then the drive module moves the elongate flexible medical instrument at the movement speed setting received in the frame which was most recently sent by remote control interface 3.

The successively sent frames of movement speed settings are numbered relative to each other, so that catheter robot 1 can reconstruct their chronology.

When the catheter robot no longer receives the frames of speed settings that it should be receiving from remote control interface 3, for a duration exceeding a first given threshold S1, then remote control interface 3 may also trigger a preliminary remote control interface alarm that is perceptible to the user of this remote control interface.

Robotic catheterization system 10 also comprises an instrumentation and control unit 4 associated with remote control interface 3 for catheter robot 1, and a robot control unit 5 associated with catheter robot 1. Control units 4 and 5 are respectively located one on either side of long-distance link 8.

Instrumentation and control unit 4, associated with remote control interface 3 for the catheter robot via a local bus 6, for example a CAN bus, comprises a converter which converts data in a local format interpretable by remote control interface 3, into packets in a long-distance format which can be sent over long-distance link 8. Instrumentation and control unit 4 also comprises a converter which converts packets in a long-distance format that are received from long-distance link 8, into data in a local format interpretable by remote control interface 3.

In order to modify the data frames, the converter of instrumentation and control unit 4 converts the frames sent by remote control interface 3 into text, then edits the text in the desired manner, and converts the modified text into a data packet for the protocol of long-distance link 8, for example the TCP-IP protocol.

Robot control unit 5 associated with catheter robot 1 via a local bus 7, for example a CAN bus, comprises a converter which converts packets in a long-distance format that are received from long-distance link 8, into data in a local format interpretable by catheter robot 1. Robot control unit 5 also comprises a converter which converts data in a local format interpretable by catheter robot 1, into packets in a long-distance format which can be sent over long-distance link 8.

The converter of robot control unit 5 converts the frames transmitted by robot 1 into text, then edits the text in the desired manner, and converts the modified text into a data packet for the protocol of long-distance link 8, for example the TCP-IP protocol.

The converters of instrumentation and control unit 4 and robot 5 are also capable of converting the TCP/IP data packet into text, editing the text, then converting the modified text into a data frame for local data bus 6 and 7, for example CAN buses.

The converter of instrumentation and control unit 4 may in particular write in the data frame transmitted by remote control interface 3, in bits not used by remote control interface 3, in order to number the data frame. The converter of robot control unit 5 can thus simultaneously read the numbers of the data frames received in order to select the frame with the highest number as being the most recent.

System 10 also comprises a medical imaging device 2 which allows checking the position of the different medical instruments being manipulated by robot 1 in the patient's body. Medical imaging device 2 comprises for example an X-ray imaging system.

In order to establish a high level of safety for the operation, monitoring of the link between remote control interface 3 and robot 1 is carried out throughout the use of robot 1 by the physician.

Verification of a good connection is carried out by remote control interface 3, which sends data frames at a predefined frequency. The frequency of transmission of data frames by remote control interface 3 may for example be 10 milliseconds for speed setting data frames coming from the physician. The software of robot 1 monitors the reception of data frames. This monitoring of the correct reception of data frames may also be performed for frames other than the physician speed setting.

Remote control interface 3 comprises a screen so that the physician has video feedback for the operation, for example including medical imaging showing the position of the medical instruments in the patient, the patient's vitals, robot 1 itself, inside the operating room.

The block instructions respectively issued by remote control interface 3 and by catheter robot 1 are accompanied by alarms respectively issued by remote control interface 3 and by catheter robot 1, which may be visual only, or audio only, or both visual and audio, and are identical or different for remote control interface 3 and for catheter robot 1.

Link 8 is a long-distance link, meaning at least 100 m or at least 1 km or at least 10 km or at least 50 km, or at least 100 km, or at least 200 km, between catheter robot 1 and remote control interface 3. Indeed, the proposed invention is all the more attractive when the risk of excessive latency increases, which is the case, in particular, for a long-distance link.

Link 8 traverses a communications or telecommunications network comprising switches and/or routers and/or servers and/or repeaters, which makes the proposed invention all the more attractive when the risk of excessive latency increases, which is the case, in particular, for a long-distance link over a complex network full of signal processing devices.

The movement speed setting may be a translational movement speed setting. The movement speed setting may also be a rotational movement speed setting. Advantageously, the movement speed setting comprises both a translational movement speed setting and rotational movement speed setting, so as to allow performing combined movements of translation and rotation.

Symmetrically, robot 1 also sends status frames from catheter robot 1 at a predetermined frequency; the software of remote control interface 3 monitors the reception of data frames. If remote control interface 3 does not receive a status frame for a predetermined waiting period, the software of remote control interface 3 triggers an alarm and sends a stop setting to robot 1, which results in the application of platform safety measures and therefore the stopping of all actuators.

More precisely, catheter robot 1 sends, at a second frequency to remote control interface 3 via link 8, status frames for catheter robot 1 which are representative of the last frames of speed settings received.

When remote control interface 3 receives these status frames, then remote control interface 3 continues to send frames of speed settings.

When remote control interface 3 no longer receives these status frames for a duration exceeding a third given threshold, then remote control interface 3 continues to send frames of speed settings.

When the remote control interface no longer receives these status frames for a duration exceeding a fourth given threshold that is greater than the third threshold, then remote control interface 3 triggers a remote interface alarm perceptible to the user of remote control interface 3.

The value of the fourth threshold is at least three times that of the third threshold.

Preferably, the third threshold is equal to the first threshold, and the fourth threshold is equal to the second threshold. Thus, the synchronization between catheter robot 1 and remote control interface 3 is further improved.

The status frames successively returned are numbered relative to each other, so that remote control interface 3 can reconstruct their chronology.

The first threshold is between 10 ms and 200 ms, or between 50 ms and 150 ms, or is 100 ms. The second threshold is between 200 ms and 5000 ms, or between 400 ms and 2000 ms, or is 1000 ms. The period of time during which the movement speed of the elongate flexible medical instrument is reduced relative to the last speed setting received, until it is canceled, is between 10 ms and 300 ms, or between 100 ms and 200 ms, or is 150 ms.

Local communication buses 6 and 7 are CAN buses. Long-distance link 8 transmits data packets in TCP/IP format or in UDP/IP format, and crosses a WAN communications network, for example the Internet.

The first frequency at which the frames of speed settings are sent by remote control interface 3, is constant and is between 1 Hz and 1 kHz, or between 10 Hz and 500 Hz, or between 50 Hz and 200 Hz, or is 100 Hz. The second frequency, at which the status frames are sent by catheter robot 1, is constant and is between 1 Hz and 1 kHz, or between 10 Hz and 500 Hz, or between 50 Hz and 200 Hz, or is 100 Hz.

FIG. 2 schematically represents a first example of a speed profile C1 as a function of time t, corresponding to a first type of reaction of catheter robot 1 following the loss of reception of the speed setting Vc sent by remote control interface 3 in an example of a robotic catheterization system 10 according to one embodiment of the invention.

When catheter robot 1 no longer receives these frames of speed settings, for a duration remaining below first given threshold S1, then the drive module continues to apply a movement speed corresponding to the last speed setting Vc received, to the elongate flexible medical instrument.

When catheter robot 1 no longer receives these frames of speed settings for a duration exceeding first given threshold S1, then the drive module reduces and cancels the movement speed of the elongate flexible medical instrument, for a duration of less than 10% of first threshold S1, as can be seen in curve C1. Thus, the movement of the elongate flexible medical instrument in the catheter robot is stopped very quickly, which further improves patient safety. More precisely, first, if robot control unit 5 has not received a data frame since first threshold S1, then robot control unit 5 controls robot 1 so that the movement speed of the various medical instruments is zero. Robot control unit 5 continues to send frames of speed settings to robot 1 so that the software of robot 1 does not yet trigger an alarm. Then, if robot control unit 5 has not received a data frame for a duration equal to a second threshold S3, robot control unit 5 can stop sending frames of speed settings to robot 1; the software of robot 1 will trigger an alarm. Robot control unit 5 thus detects a network problem due to the non-reception of frames coming from remote control interface 3 since first threshold S1, and on the one hand ensures the safety of the patient by generating frames of speed settings for robot 1 to slow down the movement of the medical instrument, and on the other hand prevents the software of robot 1 from triggering an alarm indicating non-reception of frames coming from remote control interface 3.

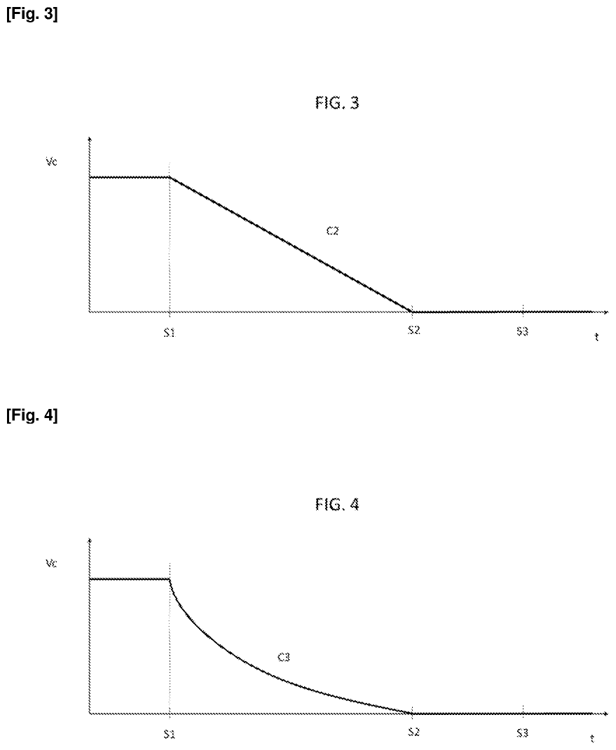

In FIGS. 3 to 5, when catheter robot 1 no longer receives these frames of speed settings for a duration exceeding first given threshold S1, then the drive module reduces and cancels the movement speed of the elongate flexible medical instrument relative to the last speed setting received, progressively for a duration S2-S1 which is more than twice first threshold S1.

In FIGS. 3 to 5, when catheter robot 1 no longer receives these frames of speed settings for a duration which remains below first given threshold S1, then the drive module continues to apply, to the elongate flexible medical instrument, a movement speed corresponding to the last speed setting Vc received.

FIG. 3 schematically represents a second example of a speed profile C2 C1 as a function of time t, corresponding to a second type of reaction of catheter robot 1 following the loss of reception of the speed setting Vc sent by remote control interface 3 in an example of a robotic catheterization system 10 according to one embodiment of the invention.

When catheter robot 1 no longer receives these frames of speed settings for a duration exceeding first given threshold S1, then the drive module reduces the movement speed of the elongate flexible medical instrument relative to the last speed setting received, progressively and linearly as can be seen in curve C2, for a duration S2-S1 which is more than twice first threshold S1. Thus, good fluidity in the operation and in the resumption of movement of the elongate flexible medical instrument in catheter robot 1 is obtained in the event that new frames of speed settings are received after a short period of not receiving these frames, while still ensuring patient safety in a satisfactory manner.

FIG. 4 schematically represents a third example of a speed profile C3 C1 as a function of time t, corresponding to a third type of reaction of catheter robot 1 following the loss of reception of the speed setting Vc sent by remote control interface 3 in an example of a robotic catheterization system 10 according to one embodiment of the invention.

When catheter robot 1 no longer receives these frames of speed settings for a duration exceeding first given threshold S1, then the drive module reduces the movement speed of the elongate flexible medical instrument relative to the last speed setting received, progressively and increasingly slower as can be seen in curve C3, advantageously in a convex parabola, for a duration S2-S1 that is more than twice first threshold S1. Thus, very good fluidity in the operation and in the resumption of movement of the elongate flexible medical instrument in catheter robot 1 is obtained in the event that new frames of speed settings are received after a short period of not receiving these frames, while still ensuring patient safety in a rather satisfactory manner.

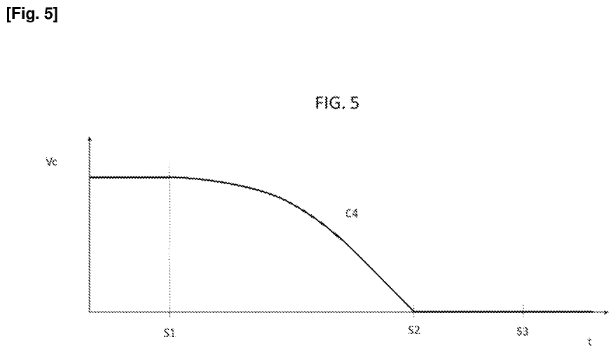

FIG. 5 schematically represents a fourth example of a speed profile C4 C1 as a function of time t, corresponding to a fourth type of reaction of catheter robot 1 following the loss of reception of the speed setting Vc sent by remote control interface 3 in an example of a robotic catheterization system 10 according to one embodiment of the invention.

When catheter robot 1 no longer receives these frames of speed settings for a duration exceeding first given threshold S1, then the drive module reduces the movement speed of the elongate flexible medical instrument relative to the last speed setting received, progressively and less and less as can be seen in curve C4, advantageously in a concave parabola, for a duration S2-S1 which is more than twice first threshold S1. Thus, fairly good fluidity in the operation and in the resumption of movement of the elongate flexible medical instrument in the catheter robot is obtained in the event that new frames of speed settings are received after a short period of not receiving these frames, while still ensuring patient safety in a very satisfactory manner.

For all of the embodiments described in FIGS. 2 to 5, if instrumentation and control unit 4 does not receive data frames transmitted by robot 1 for a predetermined duration, instrumentation and control unit 4 transmits data frames identical to the last one received by robot 1, in order to prevent the software of remote control interface 3 from sending an instruction to stop robot 1 too early and triggering an alarm prematurely. According to one possible option, if instrumentation and control unit 4 has not received a data frame for a duration equal to a second threshold S3, instrumentation and control unit 4 may stop sending data frames to remote control interface 3, and remote control interface 3 will then trigger an alarm after a predetermined duration (for example 200 ms) after the last frame received from instrumentation and control unit 4.

According to one embodiment which allows improving safety, catheter robot 1 and remote interface 3 both regularly measure the latency of link 8 by measuring the time a message takes to make the round trip between catheter robot 1 and remote interface 3. This measurement is usually called a “ping”. When catheter robot 1 or remote interface 3 detect that the measured latency exceeds a predetermined value, catheter robot 1 blocks the movement of the medical instrument in catheter robot 1, advantageously by entering robot error mode, which will require entering a first release instruction preferably directly at the local control interface before being able to continue the movement of the elongate flexible medical instrument in catheter robot 1.

To do this, catheter robot 1 sends a latency measurement message to remote interface 3 at a third frequency, while remote interface 3 also sends a latency measurement message to catheter robot 1 at said third frequency. The third frequency advantageously corresponds to a period of between 10 ms and 200 ms, for example between 50 ms and 150 ms, and is for example 100 ms. Upon the reception by remote interface 3 of the latency measurement message sent by catheter robot 1, remote interface 3 sends said latency measurement message back to catheter robot 1. Similarly, upon the reception by catheter robot 1 of the latency measurement message sent by remote interface 3, catheter robot 1 sends said latency measurement message back to remote interface 3. Catheter robot 1 measures the duration between transmission and reception of the latency measurement message by said catheter robot 1, and in a similar manner, remote interface 3 measures the duration between transmission and reception of the latency measurement message by remote interface 3. When the ping, meaning the duration between transmission and reception of the latency measurement message by catheter robot 1 or by remote interface 3, reaches a predetermined value then the drive module of catheter robot 1 blocks the movements of the medical instrument, and action on catheter robot 1 is necessary to resume operation. The predetermined value above which the drive module of catheter robot 1 blocks the medical instrument is advantageously between 400 ms and 10000 ms, preferably between 800 ms and 4000 ms, and preferably is equal to 2000 ms.

According to one possible variant, only catheter robot 1 measures the latency by pinging remote interface 3.

Of course, the invention is not limited to the examples and the embodiment described and represented, but is capable of numerous variants accessible to those skilled in the art.

1. Robotic catheterization system comprising:
a catheter robot (1) comprising a drive module for an elongate flexible medical instrument and a local control interface for the catheter robot (1),
a remote control interface (3) for the catheter robot (1),
a communication link (8) between the catheter robot (1) and the remote control interface (3),
the remote control interface (3) enabling a practitioner to control the movement speed of the elongate flexible medical instrument in the catheter robot (1) by means of the link (8),
characterized in that:
the remote control interface (3) sends, at a first frequency, frames of movement speed settings to the catheter robot (1) via the link (8),
when the catheter robot (1) receives these frames,
then the drive module moves the elongate flexible medical instrument at the movement speed setting received in these frames,
when the catheter robot (1) no longer receives these frames,
for a duration exceeding a first given threshold (S1),
then the drive module reduces or cancels the movement speed of the elongate flexible medical instrument relative to the last speed setting received (Vc),
for a duration exceeding a second given threshold (S3) that is greater than the first threshold (S1), then:
the drive module stops and blocks the movement of the elongate flexible medical instrument,
this movement of the elongate flexible medical instrument requiring, in order to be restarted after blocking:
a first release instruction received from the remote control interface (3),
and a second release instruction received from the local control interface of the catheter robot (1),
the value of the second threshold (S3) being at least twice that of the first threshold (S1), or at least three times that of the first threshold (S1).

2. Robotic catheterization system according to claim 1, wherein:
when the catheter robot (1) no longer receives these frames,
for a duration exceeding a first given threshold (S1),
then the remote control interface (3) also triggers a remote control interface alarm perceptible to the user of this remote control interface (3).

3. Robotic catheterization system according to claim 1, wherein:
when the catheter robot (1) no longer receives these frames,
for a duration exceeding the first given threshold (S1),
then the drive module reduces and cancels the movement speed of the elongate flexible medical instrument, for a duration that is less than 10% of the first threshold.

4. Robotic catheterization system according to claim 1, wherein:
when the catheter robot (1) no longer receives these frames,
for a duration exceeding the first given threshold (S1),
then the drive module reduces the movement speed of the elongate flexible medical instrument relative to the last speed setting received (Vc), progressively for a duration (S2-S1) that is greater than the first threshold (S1) or greater than twice the first threshold (S1).

5. Robotic catheterization system according to claim 4, wherein:
when the catheter robot (1) no longer receives these frames,
for a duration exceeding the first given threshold (S1),
then the drive module reduces and cancels the movement speed of the elongate flexible medical instrument relative to the last speed setting received (Vc), progressively for a duration (S2-S1) that is greater than the first threshold (S1) or greater than twice the first threshold (S1).

6. Robotic catheterization system according to claim 1, wherein:
the catheter robot (1) sends status frames for the catheter robot (1) that are representative of the last frames of speed settings received, to the remote control interface (3) at a second frequency, via the link (8),
when the remote control interface (3) receives these status frames,
then the remote control interface (3) continues to send frames of speed settings,
when the remote control interface (3) no longer receives these status frames,
for a duration exceeding a third given threshold,
then the remote control interface (3) continues to send frames of speed settings,
for a duration exceeding a fourth given threshold that is greater than the third threshold, then:
the remote control interface (3) triggers a remote interface alarm perceptible to the user of the remote control interface (3),
the value of the fourth threshold being at least twice that of the third threshold, or at least three times that of the third threshold.

7. Robotic catheterization system according to claim 1, wherein:
the third threshold is equal to the first threshold (S1),
and/or the fourth threshold is equal to the second threshold (S3).

8. Robotic catheterization system according to claim 1, the link (8) being a long-distance link, meaning at least 100 m or at least 1 km or at least 10 km or at least 50 km, between the catheter robot (1) and the remote control interface (3), characterized in that it also comprises:
an instrumentation and control unit (4) associated with the remote control interface (3) for the catheter robot (1), comprising:
a converter which converts data in a local format interpretable by the remote control interface (3) into packets in a long-distance format which can be sent over the long-distance link (8),
a robot control unit (5) associated with the catheter robot (1), comprising:
a converter which converts packets in a long-distance format that are received from the long-distance link (8) into data in a local format interpretable by the catheter robot (1),
the instrumentation and control unit (4) and the robot control unit (5) respectively being located at the two ends of the long-distance link (8).

9. Robotic catheterization system according to claim 1, wherein the second threshold (S3) is chosen such that the elongate flexible medical instrument cannot travel more than 10 mm in translational movement, after having stopped receiving the frames of speed settings, even at its maximum speed of translational movement.

10. Robotic catheterization system according to claim 1, wherein the second threshold (S3) is chosen such that the elongate flexible medical instrument cannot rotate more than 360° after have stopped receiving the frames of speed settings, even at its maximum speed of rotational movement.

11. Robotic catheterization system according to claim 1, wherein:
when the catheter robot (1) once again receives the frames, after no longer receiving these frames for some time,
the drive module then moves the elongate flexible medical instrument at the movement speed setting received in the frame sent most recently by the remote control interface (3).

12. Robotic catheterization system according to claim 1, wherein, after the first threshold (S1) has been exceeded, the remote control interface must return to the zero speed setting by an action by the user of the remote control interface (3), to enable the remote control interface (3) to regain control of the catheter robot (1).

13. Robotic catheterization system according to claim 1, wherein the catheter robot (1) is configured to send a latency measurement message at a third frequency to the remote interface (3) via the link (8), said remote interface (3) being configured to send said latency measurement message back to the catheter robot (1) upon receipt, said catheter robot (1) being configured to measure the duration between transmission and reception of the latency measurement message by said catheter robot (1), said catheter robot (1) being configured so that the drive module stops and blocks the movement of the elongate flexible medical instrument when the duration between transmission and reception of the latency measurement message by said catheter robot (1) exceeds a predetermined value.

14. Catheter robot of a robotic catheterization system:
comprising a drive module for an elongate flexible medical instrument and a local control interface for the catheter robot (1),
and being configured to be controlled by a remote control interface (3) for the catheter robot (1) enabling a practitioner to control the movement speed of the elongate flexible medical instrument in the catheter robot (1) by means of a communication link (8) between the catheter robot (1) and the remote control interface (3),
characterized in that:
when the catheter robot (1) receives frames of movement speed settings from the remote control interface (3) via the link (8),
then the drive module moves the elongate flexible medical instrument at the movement speed setting received in these frames,
when the catheter robot (1) no longer receives these frames,
for a duration exceeding a first given threshold (S1),
then the drive module reduces or cancels the movement speed of the elongate flexible medical instrument relative to the last speed setting received (Vc),
for a duration exceeding a second given threshold (S3) that is greater than the first threshold (S1), then:
the drive module stops and blocks the movement of the elongate flexible medical instrument,
this movement of the elongate flexible medical instrument requiring, in order to be restarted after blocking:
a first release instruction received from the remote control interface (3),
and a second release instruction received from the local control interface of the catheter robot (1),
the value of the second threshold (S3) being at least twice that of the first threshold (S1), or at least three times that of the first threshold (S1).

15. Control interface for a robotic catheterization system comprising a catheter robot (1) comprising a drive module for an elongate flexible medical instrument:
configured to enable a practitioner to control the movement speed of the elongate flexible medical instrument in the catheter robot (1) by means of a link (8) between the catheter robot (1) and the control interface (3), the control interface being intended to be remote from the catheter robot (1),
characterized in that the remote control interface (3):
sends frames of movement speed settings to the catheter robot (1), at a first frequency, via the link (8),
receives, from the catheter robot at a second frequency, via the link (8), status frames representative of the frames of speed settings received,
so that, when the remote control interface (3) receives these status frames,
then the remote control interface (3) continues to send frames of speed settings,
and so that, when the remote control interface (3) no longer receives these status frames,
for a duration exceeding a third given threshold,
then the remote control interface (3) continues to send frames of speed settings,
for a duration exceeding a fourth given threshold that is greater than the third threshold,
then the remote control interface (3) triggers a remote interface alarm perceptible to the user of the remote control interface (3),
the value of the fourth threshold being at least twice that of the third threshold, or at least three times that of the third threshold.

16. Method for operating a robotic catheterization system (10) comprising a catheter robot (1) which comprises a drive module for an elongate flexible medical instrument and a local control interface for the catheter robot (1), and comprising a remote control interface (3) for the catheter robot (1) that is connected to the catheter robot (1) via a link (8), such that the remote control interface (3) enables a practitioner to control the movement speed of the elongate flexible medical instrument in the catheter robot (1) by means of the link (8), characterized in that:
the remote control interface (3) sends frames of movement speed settings to the catheter robot (1) at a first frequency, via the link (8),
when the catheter robot (1) receives these frames,
then the drive module moves the elongate flexible medical instrument at the movement speed setting received in these frames,
when the catheter robot (1) no longer receives these frames,
for a duration exceeding a first given threshold (S1),
then the drive module reduces or cancels the movement speed of the elongate flexible medical instrument relative to the last speed setting received (Vc),
for a duration exceeding a second given threshold (S3) that is greater than the first threshold (S1), then:
the drive module stops and blocks the movement of the elongate flexible medical instrument,
this movement of the elongate flexible medical instrument requiring, in order to be restarted after blocking:
a first release instruction received from the remote control interface (3),
and a second release instruction received from the local control interface of the catheter robot (1),
the value of the second threshold (S3) being at least twice that of the first threshold (S1), or at least three times that of the first threshold (S1).
